(19) United States
(12) Patent Application Publication
(10) Pub. No.: US 2010/0313285 A1
(43) Pub. Date: Dec. 9, 2010

(54) Altering High Density Lipoprotein Levels Through UDP-N-Acetyl-Alpha-D-Galactosamine: Polypeptide N-Acetlgalactosaminyltransferase (GALNT) Modulation

(75) Inventors: Beverly Paigen, Bar Harbor, ME (US); Xiaosong Wang, Cambridge, MA (US)

Correspondence Address: NOVARTIS INSTITUTES FOR BIOMEDICAL RESEARCH, INC., 220 MASSACHUSETTS AVENUE, CAMBRIDGE, MA, 02139, US

(73) Assignee: NOVARTIS AG and The Jackson Laboratory

(21) Appl. No.: 12/747,616

(22) PCT Filed: Dec. 16, 2008
(86) PCT No.: PCT/EP2008/067636

§ 371 Date: Aug. 24, 2010

Related U.S. Application Data
(60) Provisional application No. 61/014,281, filed on Dec. 17, 2007.

(51) Int. Cl. A01K 67/033; C07K 16/28; A61K 39/395; C12Q 1/68; C12Q 1/48; A61P 3/04; A61P 3/06; A61P 3/10; A61P 9/10; A61P 13/12; A61P 25/28; A61K 31/713

(52) U.S. Cl. 800/13; 530/389.1; 424/172.1; 435/6; 435/15; 514/44 A

(57) ABSTRACT

Described herein are methods for detecting and treating coronary artery disease and atherosclerotic conditions based on modulating the levels of total plasma lipoprotein and HDL-C by inhibiting expression or activity of GALNT. Also described herein are methods for identifying an agent(s) useful in treating coronary artery disease or atherosclerotic conditions.

### FIELD OF THE INVENTION

The present invention relates generally to methods of treating a coronary artery disease or atherosclerotic condition. In particular, the methods comprise inhibiting the expression and/or activity of a UDP-N-acetyl-alpha-D-galactosamine:polypeptide N-acetylgalactosaminyltransferase (GALNT) to increase high-density lipoprotein levels.

### BACKGROUND OF THE INVENTION

Atherosclerosis is characterized by lipid accumulation, inflammatory response, cell death, and fibrosis in the arterial wall. Atherosclerotic disease is the leading cause of morbidity and mortality, particularly in the United States and in Western European countries. Many risk factors are implicated in the development of atherosclerosis, including those that are genetically controlled, such as family history, high plasma low-density lipoprotein (LDL) levels and low plasma high-density lipoprotein (HDL) levels, hypertension, diabetes, old age, male sex, and lifestyle factors such as smoking, consuming fatty and overly processed food, and physical inactivity.

Because LDL-C is proatherogenic, statin drugs were developed to lower plasma LDL-C levels and reduce the risk of adverse cardiovascular events. Recent studies suggest that reducing LDL-C levels to below current guideline targets further inhibits atherogenesis and reduces adverse coronary events. Statin drugs have reduced new adverse cardiovascular events by one-third; although this is significant, it is clear that additional therapies are needed. Although there is evidence that increasing plasma HDL cholesterol (HDL-C) levels inhibits atherogenesis, there do not appear to be many effective HDL-raising drugs in existence. Accordingly, a need exists to identify agents that increase HDL levels.

### SUMMARY OF THE INVENTION

The present invention is directed to the novel finding that UDP-N-acetyl-alpha-D-galactosamine:polypeptide N-acetylgalactosaminyltransferase (herein “GALNT”) regulates the level of high density lipoprotein levels in plasma. The role of this protein in affecting plasma lipoprotein levels was previously unknown. This invention shows that GALNT affects plasma levels of HDL cholesterol; specifically, GALNT activity inhibition can be used to raise plasma HDL cholesterol levels, and treat atherosclerotic disease.

Accordingly, in one aspect, the invention pertains to use of an isolated antibody or functional fragment thereof, comprising an antigen-binding region that is specific for an epitope of a polypeptide encoded by a GALNT gene (e.g., a human GALNT2 gene), wherein the antibody or functional fragment binds to a surface receptor on a cell, and prevents or ameliorates development of a HDL-associated disease.

In one embodiment, the invention pertains to a method for treating a HDL-associated disease comprising administering to a subject an effective amount of the antibody or functional fragment thereof.

The antibodies can be formulated into a pharmaceutical composition comprising an antibody or functional fragment and a pharmaceutically acceptable carrier or excipient therefore. The pharmaceutical composition can be used as a method for treating a HDL-associated disease by administering to a subject in need thereof an effective amount of the pharmaceutical composition comprising the antibody or functional fragment.

The invention also pertains to use of an isolated antibody or functional fragment thereof for the preparation of a medicament for the treatment of a HDL-associated disease, wherein the antibody or functional fragment comprising an antigen-binding region that is specific for an epitope of a polypeptide encoded by a GALNT gene (e.g., a human GALNT2 gene). Also within the scope of the invention are transgenic animals carrying a gene encoding an antibody or functional fragment.

In one embodiment, the invention is directed to a method for treating a coronary artery disease or atherosclerotic condition comprising inhibiting the expression of a GALNT. In a particular embodiment, the GALNT is human GALNT2.

In another embodiment, the invention is directed to a method for detecting a coronary artery disease or susceptibility to a coronary artery disease comprising detecting alleles of the human GALNT2 gene that are associated with the risk of coronary artery disease or an atherosclerotic condition.

In another embodiment, the invention is directed to a method for determining the efficacy of treating a coronary artery disease or atherosclerotic condition and comparing the level of the GALNT in a biological sample with a reference such that the efficacy of treating the coronary artery disease or atherosclerotic condition is determined. In a particular embodiment, the GALNT is human GALNT2.

In another embodiment, the invention is directed to a method of identifying an agent useful for treating a coronary artery disease or an atherosclerotic condition, wherein inhibition of the GALNT induces increased plasma HDL-C levels comprising contacting a biological sample with a candidate agent and determining the level of HDL-C in the sample before and after contact with the candidate agent, wherein an increase in HDL levels is indicative of an agent that is useful for treating a coronary artery disease or an atherosclerotic condition.

In another embodiment, the invention is directed to a method for identifying an agent useful for treating a coronary artery disease or atherosclerotic condition comprising contacting a GALNT with a candidate agent in the presence of a known GALNT substrate, wherein a decrease in the activity of the GALNT identifies the candidate agent as an agent useful for treating a coronary artery disease or an atherosclerotic condition. In a particular embodiment, the GALNT is human GALNT2. In a particular embodiment, the contacting step is performed in cultured cells. In another embodiment, the contacting step is performed in vivo. In another embodiment, the GALNT is endogenous or exogenous.

In another embodiment, the invention is directed to a method for modulating an HDL-associated disease comprising administering a HDL modulating agent that elevates HDL levels in a subject. In a particular embodiment, the HDL-associated disease is selected from the group consisting of: atherosclerotic cardiovascular disease (coronary artery disease, stroke, heart failure, peripheral artery disease), lipid disorders, Alzheimer's disease, excessive oxidative stress, endothelial dysfunction, obesity, chronic renal disease, diabetes and insulin resistance. The lipid disorders can be, for example, elevated plasma cholesterol levels, dyslipidemic syndrome, elevated triglycerides, dyslipidemia, dyslipoproteinemia, hyperlipidemia, familial hypercholesterolemia, and familial hypertriglyceridemia. In another embodiment, the modulating agent is selected from the group consisting of a small molecule, an antisense oligonucleotide, siRNA, shRNA and an antibody. In a particular embodiment, the HDL-associated disease is any disease in which the HDL-C levels in the subject is below the accepted normal HDL-C level. In a particular embodiment, the HDL-associated disease is any disease in which the HDL-C levels in the subject is below the accepted normal HDL-C level of the related population. In another embodiment, the agent is administered with a pharmaceutically acceptable carrier.

### BRIEF DESCRIPTION OF THE FIGURES

FIG. 1 presents charts showing the mRNA expression level of GALNT2 in various human (FIG. 1A) and mouse (FIG. 1B) tissues relative to internal controls (β-actin RNA for mouse tissues and 18s RNA for human tissues)

### DETAILED DESCRIPTION

The present invention relates to the unexpected finding that GALNT2 expression correlates to plasma levels of HDL cholesterol. Such inhibition raises plasma HDL-C levels. These enzymes belong to the family of glycosyltransferases, specifically the hexosyltransferases, which are involved in the synthesis of oligosaccharides, polysaccharides, and glycoconjugates (Breton et al., 2006, Glycobiology 16:29R-37R). UDP-N-acetyl-alpha-D-galactosamine:polypeptide N-acetylgalactosaminyltransferase (EC 2.4.1.41, family 27 in CAZy) initiates synthesis of mucin-type glycoproteins by transferring an N-acetyl galactosamine (GalNAc) to the hydroxyl group of a serine or threonine residue in the first step of O-linked oligosaccharide biosynthesis. The family has 20 members in humans, which are evolutionarily conserved. The substrate preferences for the individual iso forms has not been determined and the precise function of GALNT2 in lipoprotein metabolism has not been elucidated.

### DEFINITIONS

As used herein, the term “HDL” refers to high-density lipoprotein. HDL comprises a complex of lipids and proteins in approximately equal amounts that functions as a transporter of cholesterol in the blood. HDL is mainly synthesized in and secreted from the liver and epithelial cells of the small intestine. Immediately after secretion, HDL is in a form of a discoidal particle containing apolipoprotein A-I (also called apoA-I) and phospholipid as its major constituents, and is also called nascent HDL. This nascent HDL receives, in blood, free cholesterol from cell membranes of peripheral cells or cholesterol produced in the course of hydrolysis of other lipoproteins. Mature spherical HDL holds, at its hydrophobic center, cholesterol ester converted from said cholesterol by the action of LCAT (lecithin cholesterol acyltransferase). HDL plays an extremely important role in a lipid metabolism process called “reverse cholesterol transport”, which takes, in blood, cholesterol out of peripheral tissues and transports it to the liver. High levels of HDL are associated with a decreased risk of atherosclerosis and coronary heart disease (CHD) as the reverse cholesterol transport is considered one of the major mechanisms for HDL's prophylactic action on atherosclerosis.

As used herein, the term “HDL modulating agent” refers to any molecule able to alter the expression or functional levels of HDL such that the alteration in the HDL levels alters a HDL associated disease or condition. The HDL modulating agent can act directly or indirectly with a nucleic acid or polypeptide that effects HDL levels. Examples of HDL modulating agents include, but are not limited to antibodies, siRNA/shRNA molecules, and low molecular weight compounds. In one embodiment, the HDL modulating agent is an isolated antibody or functional fragment thereof comprising an antigen-binding region that is specific for an epitope of a polypeptide encoded by a CES gene (e.g., a hCES1 gene). The antibody or functional fragment can bind to a surface receptor on a cell, and prevent or ameliorate development of a HDL-associated disease.

As used herein, the term “biological sample” refers to a whole organism or a subset of its tissues, cells or component parts (e.g., body fluids, including but not limited to blood, mucus, lymphatic fluid, synovial fluid, cerebrospinal fluid, saliva, amniotic fluid, amniotic cord blood, urine, vaginal fluid and semen). A “biological sample” further refers to a homogenate, lysate or extract prepared from a whole organism or a subset of its tissues, cells or component parts, or a fraction or portion thereof, including, but not limited to, plasma, serum, spinal fluid, lymph fluid, the external sections of the skin, respiratory, intestinal, and genitourinary tracts, tears, saliva, milk, blood cells, tumors, organs. Most often, the sample has been removed from an animal, but the term “biological sample” can also refer to cells or tissue analyzed in vivo, e.g., without removal from animal. Typically, a “biological sample” will contain cells from the animal, but the term can also refer to non-cellular biological material, such as non-cellular fractions of blood, saliva, or urine, which can be used to measure the disease-associated polynucleotide or polypeptides levels. A “biological sample” further refers to a medium, such as a nutrient broth or gel in which an organism has been propagated, which contains cellular components, such as proteins or nucleic acid molecules.

As used herein, the term “nucleic acid” refers to deoxyribonucleotides or ribonucleotides and polymers thereof in either single- or double-stranded form. The term encompasses a nucleic acid containing known nucleotide analogs or modified backbone residues or linkages, which are synthetic, naturally occurring, and non-naturally occurring, which have similar binding properties as the reference nucleic acids, and which are metabolized in a manner similar to the reference nucleotides. Examples of such analogs include, without limitation, phosphorothioates, phosphoramidates, methyl phosphonates, chiral-methyl phosphonates, 2-O— methyl ribonucleotides, and peptide-nucleic acids (PNAs). A nucleic acid sequence also encompasses naturally-occurring allelic variants of said nucleic acid.

As used herein, the term “oligonucleotide” refers to a nucleic acid molecule consisting of two or more deoxyribonucleotides or ribonucleotides joined by phosphodiester bonds, and preferably containing between about 6 and about 300 nucleotides in length. The size of the oligonucleotide will depend on many factors, including the ultimate function or use of the oligonucleotide. Preferably, an oligonucleotide that functions, for example, as an extension primer will be sufficiently long to prime the synthesis of extension products in the presence of a catalyst, e.g., DNA polymerase, and deoxynucleotide triphosphates. As used herein, the term “oligonucleotide” further refers to an oligonucleotide that has been modified structurally (“modified oligonucleotide”) but functions similarly to the unmodified oligonucleotide. A modified oligonucleotide can contain non-naturally occurring portions, such as altered sugar moieties or inter-sugar linkages, such as a phosphorothioate.

As used herein, the term “polypeptide” refers to a polymer in which the monomers are amino acids and are joined together through peptide or disulfide bonds. It also refers to either a full-length naturally-occurring amino acid sequence or a fragment thereof between about 8 and about 500 amino acids in length. Additionally, unnatural amino acids, for example, beta-alanine, phenyl glycine and homoarginine can be included. All of the amino acids used in the present invention can be either the D- or L-optical isomer. A polypeptide sequence also encompasses naturally-occurring allelic variants of said polypeptide.

As used herein, the term “subject” includes any human or nonhuman animal. Preferably, the animal is a mammal, either human or non-human. A subject refers to, for example, primates (e.g., monkeys, apes and humans), cows, pigs, sheep, goats, horses, dogs, cats, rabbits, rats, mice, fish, birds and the like. In a preferred embodiment, the subject is a human.

As used herein, the term “efficacy” refers to the degree to which a desired effect is obtained. Specifically, the term refers to the degree to which plasma lipoprotein and HDL levels are modulated (e.g., elevated, increased, inhibited, reduced, or delayed). The term “efficacy” as used in the context of the present invention, also refers to relief or reduction of one or more symptoms or clinical events of a coronary artery disease (CAD). Relief or reduction of the symptoms include, but are not limited to, a reduction or elimination of phosphatidylcholine degradation, oxidized phospholipids, a reduction in atherosclerotic plaque formation and rupture, a reduction in clinical events such as heart attack, angina, or stroke, a decrease in hypertension, a decrease in inflammatory mediator biosynthesis, reduction in plasma cholesterol, and the like. Relief or reduction of the symptoms can also refer to improving blood flow to vascular beds affected by atherosclerosis.

As used herein, the term “coronary artery disease” (CAD) or interchangeably “coronary heart disease” (CHD) refers to a cardiovascular disease characterized by blockage of the coronary arteries. Blockage can occur suddenly, by mechanisms such as plaque rupture or embolization. Blockage can occur progressively, with narrowing of the artery via myointimal hyperplasia and plaque formation. As a plaque thickens, the artery narrows and blood flow decreases, which results in a decrease in oxygen to the myocardium. This decrease in blood flow precipitates a series of consequences for the myocardium. For example, interruption in blood flow to the myocardium results in an “infarct” (myocardial infarction), which is commonly known as a heart attack. Those clinical signs and symptoms resulting from the blockage of arteries serving the heart are manifestations of CAD. Manifestations of CAD include angina, ischemia, myocardial infarction, cardiomyopathy, congestive heart failure, arrhythmias and aneurysm formation. It is understood that fragile plaque disease in the coronary circulation is associated with arterial thrombosis or distal embolization that manifests itself as a myocardial infarction. The CAD can cover a spectrum of disease stages. The early stage of the CAD is characterized with atheromatous streaks within the walls of the coronary arteries that do not obstruct the flow of blood. Over a period of years, these streaks increase in thickness. Thus, the next stage of CAD is characterized by the formation of plaques that expand into the walls of the arteries and the lumen of the vessel and affect the blood flow through the arteries. As the plaques grow in thickness and obstruct the majority of the diameter of the vessel, the subject may have symptoms of obstructive CAD or ischemic heart disease. The symptoms often include exertional angina or decreased exercise tolerance. As the degree of the CAD progresses, there can be near-complete obstruction of the lumen of the coronary artery, severely restricting the flow of oxygen-carrying blood to the myocardium. This stage of CAD is called myocardial infarction (heart attack), and is characterized with signs and symptoms of chronic coronary ischemia, including symptoms of angina at rest and flash pulmonary edema.

As used herein, the term “atherosclerosis” refers to a process that leads to abnormal accumulation of cholesterol and cholesteryl esters and related lipids in macrophages, smooth muscle cells and in extracellular space, leading to narrowing and/or occlusion of one or more arteries, arterioles and transplanted veins of the body and bodily organs, including, but not limited to, the coronary arteries, aorta, renal arteries, carotid arteries, arteries supplying blood to the limbs and central nervous system, and grafted veins in by-pass surgery. The atherosclerosis can be quite insidious lasting for decades until atherosclerotic lesion, through physical forces from blood flow, becomes disrupted and arterial wall components are exposed to flowing blood, leading to thrombosis and compromised oxygen supply to target organs such as heart or brain.

As used herein, “HDL-associated disease” refers to any disease or trait with low HDL levels or any disease or trait that could benefit from elevated HDL levels, e.g., atherosclerosis. These associated diseases can include, for example, atherosclerotic cardiovascular disease (coronary artery disease, stroke, heart failure, peripheral artery disease), lipid disorders, Alzheimer's disease, excessive oxidative stress, chronic renal disease, obesity, type II diabetes and insulin resistance and the so called “metabolic syndrome” which includes hypertension, dyslipidemia, central obesity, and increased fasting plasma glucose. Lipid disorders can include, for example, elevated cholesterol (LDL levels of more than 130 milligrams per deciliter, or mg/dL), Dyslipidemic syndrome, elevated triglycerides (triglyceride level as high as 1,500 mg/dL), dyslipidemia, or dyslipoproteinemia (HDL is less than 35 mg/dL), hyperlipidemia or high cholesterol, familial hypercholesterolemia (a genetic disorder that increases total and LDL cholesterol), and familial hypertriglyceridemia (inherited high triglycerides).

As used herein, the term “a significant change in the expression level” refers to either an increase or a decrease of the expression level from the control level by an amount greater than the standard error of the assay employed to assess expression. The term also refers to a change by preferably at least about 10%, about 20%, about 25%, about 30%, preferably at least about 40%, about 50%, more preferably at least about 60%, about 70%, or about 90%, about 100%, about 150%, or about 200%, or greater.

As used herein, the term “gene” refers to a nucleic acid sequence that encodes and regulates expression of a polypeptide. A gene includes, therefore, regulatory elements, e.g., promoters, splice sites, enhancers, repressor binding sites, etc. A gene can have many different “alleles,” which are sequence variations that can affect the polypeptide sequence or expression level, or have no effect on the polypeptide. A gene can include one or more “open reading frames,” which are nucleic acid sequences that encode a contiguous polypeptide. A gene can be present either endogenously or exogenously.

As used herein, the term “expression level of GALNT (or GALNT2)” refers to the amount of mRNA transcribed from the corresponding gene that is present in a biological sample. The expression level can be detected with or without comparison to a level from a control sample or a level expected of a control sample.

As used herein, the term “control level” refers to a standard level of a biomarker by which a change is measure against. In one embodiment, the “control level” can be a normal level of an expressed biomarker nucleic acid or polypeptide, or a biomarker biological activity from normal or healthy cells, tissues, or subjects, or from a population of normal or healthy cells, tissues, or subjects. By way of non-limiting example, a control level can be levels of mouse or human GALNT2 polypeptide or biological activity in a normal cell, tissue, or subjects, or plasma total lipoprotein or HDL levels.

As used herein, the term “control expression level of GALNT (or GALNT2)” refers to the amount of mRNA transcribed from the corresponding gene that is present in a biological sample representative of healthy subjects. The term “control expression level” can also refer to an established level of mRNA representative of the healthy population that has been previously established based on measurement from healthy subjects.

As used herein, “detecting” refers to the identification of the presence or absence of a molecule in a sample. Where the molecule to be detected is a polypeptide, the step of detecting can be performed, for example, by binding the polypeptide to an antibody that is detectably labeled. A detectable label is a molecule that is capable of generating, either independently, or in response to a stimulus, an observable signal. A detectable label can be, but is not limited to a fluorescent label, a chromogenic label, a luminescent label, or a radioactive label. Methods for “detecting” a label include, for example, quantitative and qualitative methods adapted for standard or confocal microscopy, FACS analysis, and those adapted for high throughput methods involving multiwell plates, arrays or microarrays. One of ordinary skill in the art can select appropriate filter sets and excitation energy sources for the detection of fluorescent emission from a given fluorescent polypeptide or dye. “Detecting” as used herein can also include the use of multiple antibodies to a polypeptide to be detected, wherein the multiple antibodies bind to different epitopes on the polypeptide to be detected. Antibodies used in this manner can employ two or more detectable labels, and can include, for example a FRET pair. A polypeptide molecule is “detected” according to the present invention when the level of detectable signal is at all greater than the background level of the detectable label, or where the level of measured polypeptide is at all greater than the level measured in a control sample.

As used herein, “detecting” also refers to identification of the presence of a target nucleic acid molecule, for example, by a process wherein the signal generated by a directly or indirectly labeled probe nucleic acid molecule (capable of hybridizing to a target in a serum sample) is measured or observed. Detection of the probe nucleic acid is directly indicative of the presence, and thus the detection, of a target nucleic acid, such as a sequence encoding a marker gene. Methods and techniques for “detecting” fluorescent, radioactive, and other chemical labels may be found in Ausubel et al. (1995, Short Protocols in Molecular Biology, 3rd Ed. John Wiley and Sons, Inc.).

Alternatively, a nucleic acid can be “indirectly detected” wherein a moiety is attached to a probe nucleic acid that will hybridize with the target, wherein the moiety comprises, for example, an enzyme activity, allowing detection of the target in the presence of an appropriate substrate, or a specific antigen or other marker allowing detection by addition of an antibody or other specific indicator. Alternatively, a target nucleic acid molecule can be detected by amplifying a nucleic acid sample prepared from a patient clinical sample, using oligonucleotide primers that are specifically designed to hybridize with a portion of the target nucleic acid sequence. Quantitative amplification methods, such as TaqMan®, can also be used to “detect” a target nucleic acid according to the invention. A nucleic acid molecule is “detected” as used herein where the level of nucleic acid measured (such as by quantitative PCR), or the level of detectable signal provided by the detectable label, is at all above the background level.

As used herein, “detecting” further refers to at least the early detection of CADs such as atherosclerosis in a subject, wherein the “early” detection refers to the detection of CADs at an early stage, preferably prior to a time when a symptom is visible. “Detecting” as used herein further refers to the detection of CADs recurrence in a subject, using the same detection criteria as indicated above. “Detecting” as used herein further refers to the measurement of a change in the degree of the CADs before and after treatment with a HDL modulating agent. In this case, a change in degree of the CADs in response to a HDL modulating agent refers to either an increase or a decrease of one or more marker genes (e.g. GALNT2) or proteins as detected by ultrasound, X-ray, endoscopy, or histology, in response to the presence of a a HDL modulating agent relative to the absence of the HDL modulating agent.

The term “antibody” as used herein refers to an intact antibody or an antigen binding fragment (i.e., “antigen-binding portion”) or single chain (i.e., light or heavy chain) thereof. An intact antibody is a glycoprotein comprising at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, CH₁, CH₂ and CH₃. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antigen binding portion” of an antibody, as used herein, refers to one or more fragments of an intact antibody that retain the ability to specifically bind to a given antigen. Antigen binding functions of an antibody can be performed by fragments of an intact antibody. Examples of binding fragments encompassed within the term “antigen binding portion” of an antibody include a Fab fragment, a monovalent fragment consisting of the V_{L}, V_{H}, C_{L} and CH₁ domains; an F(ab)₂ fragment, a bivalent fragment comprising two Fab fragments (generally one from a heavy chain and one from a light chain) linked by a disulfide bridge at the hinge region; an Fd fragment consisting of the V_{H} and CH₁ domains; an Fv fragment consisting of the V_{L} and V_{H} domains of a single arm of an antibody; a single domain antibody (dAb) fragment (Ward et al., 1989 Nature 341:544-546), which consists of a V_{H} domain; and an isolated complementarity determining region (CDR).

Furthermore, although the two domains of the Fv fragment, V_{L} and V_{H}, are coded for by separate genes, they can be joined, using recombinant methods, by an artificial peptide linker that enables them to be made as a single protein chain in which the V_{L} and V_{H} regions pair to form monovalent molecules (known as single chain Fv (scFv); see, e.g., Bird et al., 1988 Science 242:423-426; and Huston et al., 1988 Proc. Natl. Acad. Sci. 85:5879-5883). Such single chain antibodies include one or more “antigen binding portions” of an antibody. These antibody fragments are obtained using conventional techniques known to those of skill in the art, and the fragments are screened for utility in the same manner as are intact antibodies.

Antigen binding portions can also be incorporated into single domain antibodies, maxibodies, minibodies, intrabodies, diabodies, triabodies, tetrabodies, v-NAR and bis-scFv (see, e.g., Hollinger and Hudson, 2005, Nature Biotechnology, 23, 9, 1126-1136). Antigen binding portions of antibodies can be grafted into scaffolds based on polypeptides such as Fibronectin type III (Fn3) (see U.S. Pat. No. 6,703,199, which describes fibronectin polypeptide monobodies).

Antigen binding portions can be incorporated into single chain molecules comprising a pair of tandem Fv segments (V_{H}-CH₁-V_{H}-CH₁) which, together with complementary light chain polypeptides, form a pair of antigen binding regions (Zapata et al., 1995 Protein Eng. 8(10):1057-1062; and U.S. Pat. No. 5,641,870).

The terms “silence” and “inhibit the expression of”, in as far as they refer to the a target gene e.g. human GALNT2, herein refer to the at least partial suppression of the expression of the human GALNT2 gene, as manifested by a reduction of the amount of mRNA transcribed from the human GALNT2 gene which may be isolated from a first cell or group of cells in which the human GALNT2 gene is transcribed and which has or have been treated such that the expression of the human GALNT2 gene is inhibited, as compared to a second cell or group of cells substantially identical to the first cell or group of cells but which has or have not been so treated (control cells). The degree of inhibition is usually expressed in terms of

$$\frac{( mRNA in control cells ) - ( mRNA in treated cells )}{( mRNA in control cells )} \cdot 100 \%$$

Alternatively, the degree of inhibition may be given in terms of a reduction of a parameter that is functionally linked to the human GALNT2 gene transcription, e.g. the amount of protein encoded by the human GALNT2 gene which is secreted by a cell, or the number of cells displaying a certain phenotype. In principle, human GALNT2 gene silencing may be determined in any cell expressing the target, either constitutively or by genomic engineering, and by any appropriate assay.

As used herein, the term “a,” “an,” “the” and similar terms used in the context of the present invention (especially in the context of the claims) are to be construed to cover both the singular and plural unless otherwise indicated herein or clearly contradicted by the context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

The present invention is based on the novel and surprising discovery that mouse and human GALNT2 regulate the levels of plasma HDL-C, the levels of which affect the development and progression of CADs. In particular, inhibition of mouse or human GALNT2 expression and/or activity increases HDL-C levels. Accordingly, one aspect of the present invention provides methods for identifying a compound that inhibits, reduces, or delays the expression or activity of mouse or human GALNT2, or a compound that treats a CAD. Another aspect of the present invention provides uses of mouse or human GALNT2 as a biomarker for monitoring the onset, progression, or regression of a CAD, or for assessing the efficacy of a compound in treating a CAD.

### Screening Assays

In one aspect, the present invention provides a method for screening (identifying) a compound that inhibits the activity of or reduces the expression of a GALNT, e.g., mouse or human GALNT2, thereby increasing the plasma level of HDL-C. The screening can be performed, for example, by contacting a compound with a biological sample containing a GALNT, e.g., mouse or human GALNT2, and monitoring the effect of the compound on the GALNT activity of mouse or human GALNT2, monitoring the expression of mouse or human GALNT2, or monitoring the effect of the compound on HDL-C, LDL-C and VLDL-C levels in the sample. The GALNT, e.g., mouse or human GALNT2, can be in the form of an endogenous or exogenous nucleic acid molecule (e.g., endogenous gene or exogenous vector comprising a suitable reading frame), or a polypeptide, or functional fragments thereof.

The effect of the compound on modulating the level of total plasma lipoprotein or HDL-C is via modulating the activity of, for example, mouse or human GALNT2. The modulation of the activity of mouse or human GALNT2 can include, but is not limited to: 1) inhibiting or preventing the polypeptide or functional fragments thereof from degrading the HDL, 2) degrading or inducing degradation of the mouse or human GALNT2 polypeptide or functional fragments thereof, 3) inactivating the biological activity of the mouse or human GALNT2 polypeptide or the functional fragments thereof; 4) reducing or inhibiting the expression of the mouse or human GALNT2 nucleic acid molecules; and 5) degrading or destabilizing the mouse or human GALNT2 nucleic acid molecules.

For purposes of determining the effects of the compound on the HDL, a parallel sample that does not receive the compound is also monitored as a control. The treated and untreated samples are then compared by any suitable phenotypic criteria, including, but not limited to, microscopic analysis, viability testing, ability to replicate, histological examination, the level of a particular RNA or polypeptide or the complex thereof, the level of enzymatic activity, and the ability of the cells to interact with other cells or compounds, etc. Differences between the treated and untreated cells indicate effects attributable to the compound. In one embodiment, the compound can be identified for inhibiting the GALNT activity or modulating the levels of HDL-C by at least about 2%, about 5%, about 10%, about 20%, about 30%, about 50%, about 70%, about 90%, about 100%, about 150%, about 200% or more. The steps of the screening method include 1) contacting the compound with a biological sample comprising HDL and a suitable GALNT, e.g., mouse or human GALNT2; 2) determining level of the HDL in the first biological sample; 3) determining level of the HDL in a second biological sample wherein the second biological sample has not been exposed to the compound; and 4) selecting the compound wherein the level of the HDL from 2) is increased as compared with the level of HDL from 3), e.g. a 1.5 fold increase.

In one embodiment, the screening assay is a cell-free assay where a cell-free biological sample containing HDL and a suitable GALNT, e.g., mouse or human GALNT2, is contacted with a compound, and the ability of the compound to modulate plasma total- and HDL-C levels is determined. Methods of measuring HDL levels are known in the art (Sugiuchi et al., Clin. Chem., 41:717-723, 1995; Izawa et al., J. Med. Pharm. Sci., 37:1385-1388, 1997).

For the cell-free screening assay described herein, the suitable GALNT, e.g., mouse or human GALNT2, polypeptide or the functional fragments thereof can be contained in the biological sample itself, or added into the biological sample from other sources. For example, the polypeptide or the functional fragments thereof can be commercially available, or purified in significant amounts from an appropriate biological source, e.g., cultured cells. Alternatively, the proteins can be recombinantly produced from an isolated gene or cDNA by expression in a suitable prokaryotic or eukaryotic expression system, and thereafter purified, as is also known in the art. Likewise, the HDL can be contained in the biological sample itself, or added into the biological sample from other sources. The HDL can be fully isolated or partially isolated. Methods of partially or completely isolating HDL are known to those of skill in the art (Havel et al., J. Clin. Invest., 43:1345-1353, 1955; Navab et al., J. Clin. Invest., 99:2005-2019, 1997; Carroll and Rudel, J. Lipid Res., 24:200-207, 1983, McNamara et al., Clin. Chem., 40:233-239, 1994, Grauholt et al., Scandinavian J. Clin. Lab. Invest., 46:715-721, 1986; Warnick et al., Clin. Chem., 28:1379-1388, 1982; Talameh et al., Clin. Chimica Acta, 158:33-41, 1986).

In another embodiment, the screening assay is an in vivo screening assay. The in vivo screening assay can be carried out in non-human animals to discover compounds that effectively inhibit, reduce, or delay degradation of HDL or affect the production of HDL component(s) in the animals. In one non-limiting example, a compound is administered to a non-human animal, optionally following a high-fat diet, at a suitable dosage for a suitable amount of time. The animal is then bled, plasma lipoproteins are isolated, and the HDL level is determined by methods known in the art. An increase in the HDL level and a decrease in total cholesterol in the animal treated with the compound compared to the HDL level in the animal not treated with the compound, indicates that the compound inhibits the activity of, for example, mouse or human GALNT2, thereby modulating the levels of total plasma lipoprotein and/or HDL-C in the animal. Preferably, the change is at least about 1.5 fold. Also preferably, the compound modulates the level of total lipoprotein and/or HDL-C in the animal by at least about 10%, about 20%, about 30%, about 50%, about 70%, about 90%, about 100%, about 150%, about 200% or more.

Optionally, prior to being administered to the animal, the compound can be pre-screened by the cell-free screening assay as described herein, or a cell-based screening assay.

In the cell-based screening assay, a cell expressing a suitable GALNT, e.g., mouse or human GALNT2, or functional fragment(s) thereof is contacted with a compound, and the ability of the compound to modulate the GALNT activity is determined. Determining the ability of the compound to modulate the GALNT activity can be accomplished by assessing the biological activity, such as catalytic/enzymatic activity of the GALNT for an appropriate substrate, by assessing the ability of the compound to bind to or interact with the GALNT, by assessing induction of a reporter gene comprising a GALNT responsive element operatively linked to a nucleic acid encoding a detectable marker, or by assessing a suitable GALNT-regulated cellular response, for example, signal transduction or protein/protein interactions. The cell can be a mammalian cell, an insect cell, a bacterial cell, or a yeast cell, etc.

Another aspect of the present invention pertains to the compound obtained from the above screening assays. The compound can be a chemical compound, an antisense oligonucleotide, a siRNA, shRNA, a non-immunoglobulin binding scaffold or an antibody.

### Antibodies

In one embodiment, the invention pertains to modulating the HDL nucleic acid and polypeptide levels by using antibodies. An antibody can include, but is not limited to, polyclonal, monoclonal, multispecific, human, humanized, or chimeric antibodies, single chain antibodies, Fab fragments, Fv fragments, F(ab′) fragments, fragments produced by a Fab expression library, anti-iodiotypic antibodies, or other epitope binding polypeptide. An antibody of the present invention can be monospecific, dispecfic, trispecific, or of greater multispecificity. Preferably, an antibody, useful in the present invention for the detection of the mouse or human GALNT2 polypeptide, is a human antibody or fragment thereof, including scFv, Fab, Fab′, F(ab′), Fd, single chain antibody, or Fv. An antibody, useful in the invention can include a complete heavy or light chain constant region, or a portion thereof, or an absence thereof. In one embodiment, an antibody useful in the invention can be a humanized antibody, in which amino acids have been replaced in the non-antigen binding regions in order to more closely resemble a human antibody, while still retaining the original binding ability. Methods for making humanized antibodies are known in the art (Teng et al., Proc. Natl. Acad. Sci. USA, 80:7308-7312, 1983; Kozbor et al., Immunology Today, 4:7279, 1983; Olsson et al., Meth. Enzymol., 92:3-16, 1982; WO 92/06193; and EP 0239400).

In some embodiments, antigen binding portions of antibodies that bind to a GALNT2 polypeptide, (e.g., V_{H} and V_{L} chains) can be “mixed and matched” to create other anti-GALNT2 binding molecules. The binding of such “mixed and matched” antibodies can be tested using binding assays (e.g., ELISAs). When selecting a V_{H} to mix and match with a particular V_{L} sequence, typically one selects a V_{H} that is structurally similar to the V_{H} it replaces in the pairing with that V_{L}. Likewise a full length heavy chain sequence from a particular full length heavy chain/full length light chain pairing is generally replaced with a structurally similar full length heavy chain sequence. Likewise, a V_{L} sequence from a particular V_{H}/V_{L} pairing should be replaced with a structurally similar V_{L} sequence. Likewise a full length light chain sequence from a particular full length heavy chain/full length light chain pairing should be replaced with a structurally similar full length light chain sequence. Identifying structural similarity in this context is a process well known in the art.

A human antibody comprises heavy or light chain variable regions or full length heavy or light chains that are “the product of” or “derived from” a particular germline sequence if the variable regions or full length chains of the antibody are obtained from a system that uses human germline immunoglobulin genes as the source of the sequences. In one such system, a human antibody is raised in a transgenic mouse carrying human immunoglobulin genes. The transgenic is immunized with the antigen of interest (e.g., an epitope of a GALNT2 polypeptide).

A human antibody that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody. A human antibody that is “the product of” or “derived from” a particular human germline immunoglobulin sequence may contain amino acid differences as compared to the germline-encoded sequence, due to, for example, naturally occurring somatic mutations or artificial site-directed mutations. However, a selected human antibody typically has an amino acid sequence at least 90% identical to an amino acid sequence encoded by a human germline immunoglobulin gene and contains amino acid residues that identify the human antibody as being human when compared to the germline immunoglobulin amino acid sequences of other species (e.g., murine germline sequences). In certain cases, a human antibody may be at least 60%, 70%, 80%, 90%, or at least 95%, or even at least 96%, 97%, 98%, or 99% identical in amino acid sequence to the amino acid sequence encoded by the germline immunoglobulin gene.

### Camelid Antibodies

Antibody proteins obtained from members of the camel and dromedary (Camelus bactrianus and Calelus dromaderius) family, including New World members such as llama species (Lama paccos, Lama glama and Lama vicugna), have been characterized with respect to size, structural complexity and antigenicity for human subjects. Certain IgG antibodies found in nature in this family of mammals lack light chains, and are thus structurally distinct from the four chain quaternary structure having two heavy and two light chains typical for antibodies from other animals. See WO 94/04678.

A region of the camelid antibody that is the small, single variable domain identified as V_{H}H can be obtained by genetic engineering to yield a small protein having high affinity for a target, resulting in a low molecular weight, antibody-derived protein known as a “camelid nanobody”. See U.S. Pat. No. 5,759,808; see also Stijlemans et al., 2004 J. Biol. Chem. 279: 1256-1261; Dumoulin et al., 2003 Nature 424: 783-788; Pleschberger et al., 2003 Bioconjugate Chem. 14: 440-448; Cortez-Retamozo et al., 2002 Int. J. Cancer 89: 456-62; and Lauwereys. et al., 1998 EMBO J. 17: 3512-3520. Engineered libraries of camelid antibodies and antibody fragments are commercially available, for example, from Ablynx, (Ghent, Belgium). As with other antibodies of non-human origin, an amino acid sequence of a camelid antibody can be altered recombinantly to obtain a sequence that more closely resembles a human sequence, i.e., the nanobody can be “humanized”. Thus the natural low antigenicity of camelid antibodies to humans can be further reduced.

The camelid nanobody has a molecular weight approximately one-tenth that of a human IgG molecule, and the protein has a physical diameter of only a few nanometers. One consequence of the small size is the ability of camelid nanobodies to bind to antigenic sites that are functionally invisible to larger antibody proteins, i.e., camelid nanobodies are useful as reagents to detect antigens that are otherwise cryptic using classical immunological techniques, and as possible therapeutic agents. Thus, yet another consequence of small size is that a camelid nanobody can inhibit as a result of binding to a specific site in a groove or narrow cleft of a target protein, and hence can serve in a capacity that more closely resembles the function of a classical low molecular weight drug than that of a classical antibody.

The low molecular weight and compact size further result in camelid nanobodies' being extremely thermostable, stable to extreme pH and to proteolytic digestion, and poorly antigenic. Another consequence is that camelid nanobodies readily move from the circulatory system into tissues, and even cross the blood-brain barrier and can treat disorders that affect nervous tissue. Nanobodies can further facilitate drug transport across the blood brain barrier. See U.S. Pat. Pub. No. 20040161738, published Aug. 19, 2004. These features combined with the low antigenicity in humans indicate great therapeutic potential. Further, these molecules can be fully expressed in prokaryotic cells such as E. coli. Also included in the scope of the present invention are camelid antibodies Accordingly, a feature of the present invention is a camelid antibody or camelid nanobody having high affinity for the GALNT2 polypeptide.

### Diabodies

Diabodies are bivalent, bispecific molecules in which V_{H} and V_{L} domains are expressed on a single polypeptide chain, connected by a linker that is too short to allow for pairing between the two domains on the same chain. The V_{H} and V_{L} domains pair with complementary domains of another chain, thereby creating two antigen binding sites (see e.g., Holliger et al., 1993 Proc. Natl. Acad. Sci. USA 90:6444-6448; Poljak et al., 1994 Structure 2:1121-1123). Diabodies can be produced by expressing two polypeptide chains with either the structure V_{H}A-V_{L}B and V_{H}B-V_{L}A (V_{H}-V_{L} configuration), or V_{L}A-V_{H}B and V_{L}B-V_{H}A (V_{L}-V_{H} configuration) within the same cell. Most of them can be expressed in soluble form in bacteria.

Single chain diabodies (scDb) are produced by connecting the two diabody-forming polypeptide chains with linker of approximately 15 amino acid residues (see Holliger and Winter, 1997 Cancer Immunol. Immunother., 45(3-4):128-30; Wu et al., 1996 Immunotechnology, 2(1):21-36). scDb can be expressed in bacteria in soluble, active monomeric form (see Holliger and Winter, 1997 Cancer Immunol. Immunother., 45(34): 128-30; Wu et al., 1996 Immunotechnology, 2(1):21-36; Pluckthun and Pack, 1997 Immunotechnology, 3(2): 83-105; Ridgway et al., 1996 Protein Eng., 9(7):617-21). A diabody can be fused to Fc to generate a “di-diabody” (see Lu et al., 2004 J. Biol. Chem., 279(4):2856-65).

### Engineered and Modified Antibodies

An antibody of the invention can be prepared using an antibody having one or more V_{H} and/or V_{L} sequences as starting material to engineer a modified antibody, which modified antibody may have altered properties from the starting antibody. An antibody can be engineered by modifying one or more residues within one or both variable regions (i.e., V_{H} and/or V_{L}), for example within one or more CDR regions and/or within one or more framework regions. Additionally or alternatively, an antibody can be engineered by modifying residues within the constant region(s), for example to alter the effector function(s) of the antibody.

One type of variable region engineering that can be performed is CDR grafting. Antibodies interact with target antigens predominantly through amino acid residues that are located in the six heavy and light chain CDRs. For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann et al., 1998 Nature 332:323-327; Jones et al., 1986 Nature 321:522-525; Queen et al., 1989 Proc. Natl. Acad. See. U.S.A. 86:10029-10033; U.S. Pat. No. 5,225,539, and U.S. Pat. Nos. 5,530,101; 5,585,089; 5,693,762 and 6,180,370).

Framework sequences can be obtained from public DNA databases or published references that include germline antibody gene sequences. For example, germline DNA sequences for human heavy and light chain variable region genes can be found in the “VBase” human germline sequence database, as well as in Kabat et al., 1991 Sequences of Proteins of Immunological Interest, Fifth Edition, U.S. Department of Health and Human Services, NIH Publication No. 91-3242; Tomlinson et al., 1992 J. Mol. Biol. 227:776-798; and Cox et al., 1994 Eur. J. Immunol. 24:827-836; the contents of each of which are expressly incorporated herein by reference.

The V_{H} CDR1, 2 and 3 sequences and the V_{L} CDR1, 2 and 3 sequences can be grafted onto framework regions that have the identical sequence as that found in the germline immunoglobulin gene from which the framework sequence is derived, or the CDR sequences can be grafted onto framework regions that contain one or more mutations as compared to the germline sequences. For example, it has been found that in certain instances it is beneficial to mutate residues within the framework regions to maintain or enhance the antigen binding ability of the antibody (see e.g., U.S. Pat. Nos. 5,530,101; 5,585,089; 5,693,762 and 6,180,370).

CDRs can also be grafted into framework regions of polypeptides other than immunoglobulin domains. Appropriate scaffolds form a conformationally stable framework that displays the grafted residues such that they form a localized surface and bind the target of interest. For example, CDRs can be grafted onto a scaffold in which the framework regions are based on fibronectin, ankyrin, lipocalin, neocarzinostain, cytochrome b, CP1 zinc finger, PST1, coiled coil, LACI-D1, Z domain or tendramisat (See e.g., Nygren and Uhlen, 1997 Current Opinion in Structural Biology, 7, 463-469).

Another type of variable region modification is mutation of amino acid residues within the V_{H} and/or V_{L} CDR1, CDR2 and/or CDR3 regions to thereby improve one or more binding properties (e.g., affinity) of the antibody of interest, known as “affinity maturation.” Site-directed mutagenesis or PCR-mediated mutagenesis can be performed to introduce the mutation(s), and the effect on antibody binding, or other functional property of interest, can be evaluated in in vitro or in vivo assays as described herein. Conservative modifications can be introduced. The mutations may be amino acid substitutions, additions or deletions. Moreover, typically no more than one, two, three, four or five residues within a CDR region are altered.

Engineered antibodies of the invention include those in which modifications have been made to framework residues within V_{H} and/or V_{L}, e.g., to improve the properties of the antibody. Typically such framework modifications are made to decrease the immunogenicity of the antibody. For example, one approach is to “backmutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “backmutated” to the germline sequence by, for example, site-directed mutagenesis or PCR-mediated mutagenesis. Such “backmutated” antibodies are also intended to be encompassed by the invention.

Another type of framework modification involves mutating one or more residues within the framework region, or even within one or more CDR regions, to remove T cell epitopes to thereby reduce the potential immunogenicity of the antibody. This approach is also referred to as “deimmunization” and is described in further detail in U.S. Pat. Pub. No. 20030153043 by Carr et al.

In addition or alternative to modifications made within the framework or CDR regions, antibodies of the invention may be engineered to include modifications within the Fc region, typically to alter one or more functional properties of the antibody, such as serum half-life, complement fixation, Fc receptor binding, and/or antigen-dependent cellular cytotoxicity. Furthermore, an antibody of the invention may be chemically modified (e.g., one or more chemical moieties can be attached to the antibody) or be modified to alter its glycosylation, again to alter one or more functional properties of the antibody.

In one embodiment, the hinge region of CH₁ is modified such that the number of cysteine residues in the hinge region is altered, e.g., increased or decreased. This approach is described further in U.S. Pat. No. 5,677,425 by Bodmer et al. The number of cysteine residues in the hinge region of CH₁ is altered to, for example, facilitate assembly of the light and heavy chains or to increase or decrease the stability of the antibody.

In another embodiment, the Fc hinge region of an antibody is mutated to decrease the biological half-life of the antibody. More specifically, one or more amino acid mutations are introduced into the CH₂—CH₃ domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In another embodiment, the antibody is modified to increase its biological half-life. Various approaches are possible. For example, U.S. Pat. No. 6,277,375 describes the following mutations in an IgG that increase its half-life in vivo: T252L, T254S, T256F. Alternatively, to increase the biological half life, the antibody can be altered within the CH₁ or CL region to contain a salvage receptor binding epitope taken from two loops of a CH₂ domain of an Fc region of an IgG, as described in U.S. Pat. Nos. 5,869,046 and 6,121,022 by Presta et al.

In yet other embodiments, the Fc region is altered by replacing at least one amino acid residue with a different amino acid residue to alter the effector functions of the antibody. For example, one or more amino acids can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

In another embodiment, one or more amino acids selected from amino acid residues can be replaced with a different amino acid residue such that the antibody has altered C1q binding and/or reduced or abolished complement dependent cytotoxicity (CDC). This approach is described in further detail in U.S. Pat. No. 6,194,551 by Idusogie et al.

In another embodiment, one or more amino acid residues are altered to thereby alter the ability of the antibody to fix complement. This approach is described further in WO 94/29351 by Bodmer et al.

In yet another embodiment, the Fc region is modified to increase the ability of the antibody to mediate antibody dependent cellular cytotoxicity (ADCC) and/or to increase the affinity of the antibody for an Fcγ receptor by modifying one or more amino acids. This approach is described further in WO 00/42072 by Presta. Moreover, the binding sites on human IgG1 for FcγRI, FcγRII, FcγRIII and FcRn have been mapped and variants with improved binding have been described (see Shields, R. L. et al., 2001 J. Biol. Chem. 276:6591-6604).

In still another embodiment, the glycosylation of an antibody is modified. For example, an aglycoslated antibody can be made (i.e., the antibody lacks glycosylation). Glycosylation can be altered, for example, to increase the affinity of the antibody for an antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. For example, EP 1,176,195 by Hang et al. describes a cell line with a functionally disrupted FUT8 gene, which encodes a fucosyl transferase, such that antibodies expressed in such a cell line exhibit hypofucosylation. PCT Pub. WO 03/035835 by Presta describes a variant CHO cell line, Lec13 cells, with reduced ability to attach fucose to Asn(297)-linked carbohydrates, also resulting in hypofucosylation of antibodies expressed in that host cell (see also Shields, R. L. et al., 2002 J. Biol. Chem. 277:26733-26740). WO 99/54342 by Umana et al. describes cell lines engineered to express glycoprotein-modifying glycosyl transferases (e.g., beta(1,4)-N acetylglucosaminyltransferase III (GnTIII)) such that antibodies expressed in the engineered cell lines exhibit increased bisecting GlcNac structures which results in increased ADCC activity of the antibodies (see also Umana et al., 1999 Nat. Biotech. 17:176-180).

Another modification of the antibodies herein that is contemplated by the invention is pegylation. An antibody can be pegylated to, for example, increase the biological (e.g., serum) half-life of the antibody. To pegylate an antibody, the antibody, or fragment thereof, typically is reacted with polyethylene glycol (PEG), such as a reactive ester or aldehyde derivative of PEG, under conditions in which one or more PEG moieties become attached to the antibody or antibody fragment. The pegylation can be carried out by an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies of the invention. See for example, EP 0 154 316 by Nishimura et al. and EP 0 401 384 by Ishikawa et al.

In addition, pegylation can be achieved in any part of an antibody by the introduction of a nonnatural amino acid. Certain nonnatural amino acids can be introduced by the technology described in Deiters et al., J Am Chem Soc 125:11782-11783, 2003; Wang and Schultz, Science 301:964-967, 2003; Wang et al., Science 292:498-500, 2001; Zhang et al., Science 303:371-373, 2004 or in U.S. Pat. No. 7,083,970. Briefly, some of these expression systems involve site-directed mutagenesis to introduce a nonsense codon, such as an amber TAG, into the open reading frame encoding a polypeptide of the invention. Such expression vectors are then introduced into a host that can utilize a tRNA specific for the introduced nonsense codon and charged with the nonnatural amino acid of choice. Particular nonnatural amino acids that are beneficial for purpose of conjugating moieties to the polypeptides of the invention include those with acetylene and azido side chains. The polypeptides containing these novel amino acids can then be pegylated at these chosen sites in the protein.

### Methods of Engineering Antibodies

Antibodies can be modified to create new antibodies by modifying full length heavy chain and/or light chain sequences, V_{H} and/or V_{L} sequences, or the constant region(s) attached thereto. For example, one or more CDR regions of the antibodies can be combined recombinantly with known framework regions and/or other CDRs to create new, recombinantly-engineered antibodies. The starting material for the engineering method is one or more of the V_{H} and/or V_{L} sequences, or one or more CDR regions thereof. To create the engineered antibody, it is not necessary to actually prepare (i.e., express as a protein) an antibody having one or more of the V_{H} and/or V_{L} sequences, or one or more CDR regions thereof. Rather, the information contained in the sequence(s) is used as the starting material to create a “second generation” sequence(s) derived from the original sequence(s) and then the “second generation” sequence(s) is prepared and expressed as a protein.

Standard molecular biology techniques can be used to prepare and express the altered antibody sequence. The antibody encoded by the altered antibody sequence(s) is one that retains one, some or all of the desired functional properties from which it is derived. The functional properties of the altered antibodies can be assessed using standard assays available in the art and/or described herein (e.g., ELISAs).

In certain embodiments of the methods of engineering antibodies of the invention, mutations can be introduced randomly or selectively along all or part of an antibody coding sequence. For example, PCT Pub. WO 02/092780 by Short describes methods for creating and screening antibody mutations using saturation mutagenesis, synthetic ligation assembly, or a combination thereof. Alternatively, WO 03/074679 by Lazar et al. describes methods of using computational screening methods to optimize physiochemical properties of antibodies.

### Non-Antibody Binding Molecules

The invention further provides binding molecules that exhibit functional properties of antibodies but derive their framework and antigen binding portions from other polypeptides (e.g., polypeptides other than those encoded by antibody genes or generated by the recombination of antibody genes in vivo). The antigen binding domains of these binding molecules are generated through a directed evolution process. See U.S. Pat. No. 7,115,396. Molecules that have an overall fold similar to that of a variable domain of an antibody (an “immunoglobulin-like” fold) are appropriate scaffold proteins. Scaffold proteins suitable for deriving antigen binding molecules include fibronectin or a fibronectin dimer, tenascin, N-cadherin, E-cadherin, ICAM, titin, GCSF-receptor, cytokine receptor, glycosidase inhibitor, antibiotic chromoprotein, myelin membrane adhesion molecule P0, CD8, CD4, CD2, class I MHC, T-cell antigen receptor, CD1, C2 and I-set domains of VCAM-1, I-set immunoglobulin domain of myosin-binding protein C, I-set immunoglobulin domain of myosin-binding protein H, I-set immunoglobulin domain of telokin, NCAM, twitchin, neuroglian, growth hormone receptor, erythropoietin receptor, prolactin receptor, interferon-gamma receptor, β-galactosidase/glucuronidase, β-glucuronidase, transglutaminase, T-cell antigen receptor, superoxide dismutase, tissue factor domain, cytochrome F, green fluorescent protein, GroEL, and thaumatin.

The antigen binding domain (e.g., the immunoglobulin-like fold) of the non-antibody binding molecule can have a molecular mass less than 10 kD or greater than 7.5 kD (e.g., a molecular mass between 7.5-10 kD). The protein used to derive the antigen binding domain is a naturally occurring mammalian protein (e.g., a human protein), and the antigen binding domain includes up to 50% (e.g., up to 34%, 25%, 20%, or 15%), mutated amino acids as compared to the immunoglobulin-like fold of the protein from which it is derived. The domain having the immunoglobulin-like fold generally consists of 50-150 amino acids (e.g., 40-60 amino acids).

To generate non-antibody binding molecules, a library of clones is created in which sequences in regions of the scaffold protein that form antigen binding surfaces (e.g., regions analogous in position and structure to CDRs of an antibody variable domain immunoglobulin fold) are randomized. Library clones are tested for specific binding to the antigen of interest and for other functions. Selected clones can be used as the basis for further randomization and selection to produce derivatives of higher affinity for the antigen.

High affinity binding molecules are generated, for example, using the tenth module of fibronectin III (10Fn3) as the scaffold. A library is constructed for each of three CDR-like loops of 10FN3 at residues 23-29, 52-55, and 78-87. To construct each library, DNA segments encoding sequence overlapping each CDR-like region are randomized by oligonucleotide synthesis. Techniques for producing selectable 10Fn3 libraries are described in U.S. Pat. Nos. 6,818,418 and 7,115,396; Roberts and Szostak, 1997 Proc. Natl. Acad. Sci. USA 94:12297; U.S. Pat. No. 6,261,804; U.S. Pat. No. 6,258,558; and Szostak et al. WO98/31700.

Non-antibody binding molecules can be produced as dimers or multimers to increase avidity for the target antigen. For example, the antigen binding domain is expressed as a fusion with a constant region (Fc) of an antibody that forms Fc-Fc dimers. See, e.g., U.S. Pat. No. 7,115,396.

### RNA Interference

An antisense oligonucleotide is a polynucleotide that is complementary to all or part of a target primary transcript (unprocessed transcript) or mRNA and blocks the expression of a target gene (U.S. Pat. No. 5,107,065; WO 9928508). The complementarity of an antisense oligonucleotide may be with any part of the specific gene transcript, e.g., at the 5′ non-coding sequence, 3′ non-coding sequence, or the coding sequence.

An siRNA refers to a small interfering RNA, which acts to degrade mRNA sequences homologous to either of the RNA strands in the duplex and can cause post-transcriptional silencing of specific genes in cells, for example, mammalian cells (including human cells) and in the body, for example, mammalian bodies (including humans). siRNA molecules can be used for inhibiting the expression of the target gene (e.g., hGALNT2) in a cell or mammal, wherein the siRNA comprises a region of complementarity to at least part of the mRNA formed in the expression of the target gene. The phenomenon of RNA interference is known in the art (Bass, Nature, 411:428-29, 2001; Elbahir et al., Nature, 411:494-98, 2001; Fire et al., Nature, 391:806-11, 1998; and WO 01/75164). The siRNAs based upon the sequences and nucleic acids encoding the gene products disclosed herein typically have fewer than 100 base pairs and can be, e.g., about 30 by or shorter, and can be made by approaches known in the art, including the use of complementary DNA strands or synthetic approaches. The siRNAs are capable of causing interference and can cause post-transcriptional silencing of specific genes in cells, for example, mammalian cells (including human cells) and in the body, for example, mammalian bodies (including humans). Exemplary siRNAs according to the present invention can have up to 29 bp, 25 bp, 22 bp, 21 bp, 20 bp, 15 bp, 10 bp, 5 by or any integer thereabout or there between. Tools for designing optimal inhibitory siRNAs include that available from DNAengine Inc. (Seattle, Wash.) and Ambion, Inc. (Austin, Tex.).

Double-stranded ribonucleic acid (dsRNA) molecules can also be used for inhibiting the expression of the target gene (e.g., hGALNT2) in a cell or mammal, wherein the dsRNA comprises an antisense strand comprising a region of complementarity which is complementary to at least a part of an mRNA formed in the expression of the target gene, and wherein the region of complementarity is less than 30 nucleotides in length, generally 19-24 nucleotides in length, and wherein the dsRNA, upon contact with a cell expressing the target gene, inhibits the expression of the target gene by at least 10%, 25%, or 40%.

The dsRNA comprises two RNA strands that are sufficiently complementary to hybridize to form a duplex structure. One strand of the dsRNA (the antisense strand) comprises a region of complementarity that is substantially complementary, and generally fully complementary, to a target sequence, derived from the sequence of an mRNA formed during the expression of the target gene, the other strand (the sense strand) comprises a region which is complementary to the antisense strand, such that the two strands hybridize and form a duplex structure when combined under suitable conditions. Generally, the duplex structure is between 15 and 30, more generally between 18 and 25, yet more generally between 19 and 24, and most generally between 19 and 21 base pairs in length. Similarly, the region of complementarity to the target sequence is between 15 and 30, more generally between 18 and 25, yet more generally between 19 and 24, and most generally between 19 and 21 nucleotides in length. The dsRNA of the invention may further comprise one or more single-stranded nucleotide overhang(s). The dsRNA can be synthesized by standard methods known in the art by use of an automated DNA synthesizer, such as are commercially available from, for example, Biosearch, Applied Biosystems, Inc.

The skilled person is well aware that dsRNAs comprising a duplex structure of between 20 and 23, but specifically 21, base pairs have been hailed as particularly effective in inducing RNA interference (Elbashir et al., EMBO 2001, 20:6877-6888).

In yet another embodiment, the dsRNA is chemically modified to enhance stability. The nucleic acids of the invention may be synthesized and/or modified by methods well established in the art, such as those described in Current Protocols in Nucleic Acid Chemistry, Beaucage, S. L. et al. (Edrs.), John Wiley & Sons, Inc., New York, N.Y., USA, which is hereby incorporated herein by reference. Chemical modifications may include, but are not limited to 2′ modifications, modifications at other sites of the sugar or base of an oligonucleotide, introduction of non-natural bases into the oligonucleotide chain, covalent attachment to a ligand or chemical moiety, and replacement of internucleotide phosphate linkages with alternate linkages such as thiophosphates. More than one such modification may be employed.

Chemical linking of the two separate dsRNA strands may be achieved by any of a variety of well-known techniques, for example by introducing covalent, ionic or hydrogen bonds; hydrophobic interactions, van der Waals or stacking interactions; by means of metal-ion coordination, or through use of purine analogues. Generally, the chemical groups that can be used to modify the dsRNA include, without limitation, methylene blue; bifunctional groups, generally bis-(2-chloroethyl)amine; N-acetyl-N′-(p-glyoxylbenzoyl)cystamine; 4-thiouracil; and psoralen. In one embodiment, the linker is a hexa-ethylene glycol linker. In this case, the dsRNA are produced by solid phase synthesis and the hexa-ethylene glycol linker is incorporated according to standard methods (e.g., Williams, D. J., and K. B. Hall, Biochem. (1996) 35:14665-14670). In a particular embodiment, the 5′-end of the antisense strand and the 3′-end of the sense strand are chemically linked via a hexaethylene glycol linker. In another embodiment, at least one nucleotide of the dsRNA comprises a phosphorothioate or phosphorodithioate groups. The chemical bond at the ends of the dsRNA is generally formed by triple-helix bonds.

In yet another embodiment, the nucleotides at one or both of the two single strands may be modified to prevent or inhibit the degradation activities of cellular enzymes, such as, for example, without limitation, certain nucleases. Techniques for inhibiting the degradation activity of cellular enzymes against nucleic acids are known in the art including, but not limited to, 2′-amino modifications, 2′-amino sugar modifications, 2′-F sugar modifications, 2′-F modifications, 2′-alkyl sugar modifications, uncharged backbone modifications, morpholino modifications, 2′-O-methyl modifications, and phosphoramidate (see, e.g., Wagner, Nat. Med. (1995) 1:1116-8). Thus, at least one 2′-hydroxyl group of the nucleotides on a dsRNA is replaced by a chemical group, generally by a 2′-amino or a 2′-methyl group. Also, at least one nucleotide may be modified to form a locked nucleotide. Such locked nucleotide contains a methylene bridge that connects the 2′-oxygen of ribose with the 4′-carbon of ribose. Oligonucleotides containing the locked nucleotide are described in Koshkin, A. A., et al., Tetrahedron Lett 1998), 54: 3607-3630) and Obika, S. et al., Tetrahedron Lett. (1998), 39: 5401-5404). Introduction of a locked nucleotide into an oligonucleotide improves the affinity for complementary sequences and increases the melting temperature by several degrees (Braasch, D. A. and D. R. Corey, Chem. Biol. (2001), 8:1-7).

### Use of GALNT2 as a Biomarker

In one aspect, the expression levels of the differentially expressed mouse or human GALNT2 genes are determined in normal and CAD cells and/or tissues. In one embodiment, the methods of determining the expression levels of the gene(s) can comprise one or more of the following steps in any effective order, e.g., contacting a biological sample with a polynucleotide probe under conditions effective for said probe to hybridize specifically to the mouse or human GALNT2 nucleic acid molecule in said sample, and detecting the presence or absence of the mouse or human GALNT2 marker gene nucleic acid in said sample. Specific alleles, comprising distinct and relevant polymorphisms are also detected.

In one embodiment, the probe is applied to the samples obtained from both the normal and CAD cells and/or tissues, and the presence of the mouse or human GALNT2 nucleic acid molecule is detected with the methods known in the art. For example, the methods of detecting the presence of the marker genes can be carried out by any effective process, e.g., by Northern blot analysis, polymerase chain reaction (PCR), reverse transcriptase PCR, RACE PCR, in situ hybridization, etc.

In another embodiment, the probe is applied to the samples obtained from both the normal and CAD cells and/or tissues, and the amount of the mouse or human GALNT2 nucleic acid is detected with the methods known in the art. Such methods can involve, e.g., contacting with probe, hybridizing, and detecting hybridized probe, but using more quantitative methods and/or comparisons to standards. The amount of hybridization between the probe and target can be determined by any suitable methods, e.g., PCR, RT-PCR, RACE PCR, Northern blot, polynucleotide microarrays, Rapid-Scan, etc., and includes both quantitative and qualitative measurements.

In another embodiment, mouse or human GALNT2-specific antibodies can be used to detect the presence of mouse or human GALNT2, or a fragment(s) thereof, in a biological sample by any method known in the art. The method can include immunoassays such as competitive and non-competitive assay systems using techniques such as Western blots, radioimmunoassays, ELISA (enzyme linked immunosorbent assay), “sandwich” immunoassays, immunoprecipitation assays, precipitation reactions, gel diffusion precipitin reactions, immunodiffusion assays, agglutination assays, complement-fixation assays, immunoradiometric assays, fluorescent immunoassays, protein A immunoassays, to name but a few. Such assays are known in the art (Ausubel et al., eds, 1994, Current Protocols in Molecular Biology, Vol. 1, John Wiley & Sons, Inc., New York, which is incorporated by reference herein in its entirety). In addition, immunoassays useful in the present invention can also include both homogeneous and heterogeneous procedures such as fluorescence polarization immunoassay (FPIA), fluorescence immunoassay (FIA), enzyme immunoassay (EIA), and nephelometric inhibition immunoassay (NIA).

In another embodiment, the level of the mouse or human GALNT2 polypeptide in a biological sample can be determined as a way of monitoring the expression level of mouse or human GALNT2. Such a method would include, for example, the steps of obtaining a biological sample, contacting the sample with an antibody specific for the mouse or human GALNT2 polypeptide or suitable epitope thereof, and determining the amount of immune complex formation with the antibody, with the amount of immune complex formation being indicative of the level of the mouse or human GALNT2 polypeptide. This determination is instructive when the GALNT polypeptide level in a biological sample obtained from a subject with a CAD is compared to the level of the GALNT in a biological sample taken from a normal subject, or in one or more samples previously or subsequently obtained from the same subject.

Determination of the amount of the GALNT polypeptide can also be correlated with progression of a CAD. The GALNT polypeptide level can be used predictably to evaluate whether a biological sample containing these peptides are predisposing for CADs, or can be used to plan a particular therapeutic regimen.

### Diagnostic Assays

The determination of a detectable increase or decrease in the expression level of a suitable, e.g., lipoprotein-modulating GALNT, e.g., mouse or human GALNT2, in a subject with a CAD as compared to a normal subject, provides a means of diagnosing or monitoring the disease status, and/or response to benefit to a therapy. Therefore, the present invention provides methods for detecting a CAD or atherosclerotic condition, or alternatively determining whether a subject is at increased risk for developing a CAD or atherosclerotic condition.

In clinical applications, human tissue samples can be screened for the presence and/or absence of a suitable GALNT-encoding nucleic acid and/or GALNT polypeptides. Such samples can comprise tissue samples, whole cells, cell lysates, or isolated nucleic acids, including, for example, needle biopsy cores, surgical resection samples, lymph node tissue, plasma, or serum. In certain embodiments, nucleic acids extracted from these samples may be amplified using techniques well known in the art. The levels GALNT and/or plasma total and/or HDL-C detected would be compared with those in a normal tissue sample.

In one embodiment, the diagnostic method comprises determining whether a subject has an increased risk for CAD by detecting the mRNA, cDNA or polypeptide level of a suitable, e.g., lipoprotein-modulating GALNT, e.g., mouse or human GALNT2. A significant change in the expression level of the GALNT in the subject compared to that in the normal healthy subject is an indication of a CAD or a susceptibility to a CAD. Preferably, the change is at least about 10%, about 20%, about 25%, about 30%, preferably at least about 40%, about 50%, more preferably at least about 60%, about 70%, or about 90%, about 100%, about 150%, or about 200%, or greater.

Alternatively, the diagnostic method can be carried out using antibodies to detect the GALNT polypeptide or the functional fragments thereof. In one embodiment, the method includes comparing level of a suitable GALNT polypeptide molecule in a biological sample from a subject with a control level of the polypeptide molecule, wherein a significant change in the level of the GALNT polypeptide is an indication of the CAD in the subject. The term “significant change” refers to a change in the amount of the polypeptide or the functional fragments thereof relative to that from a biological sample of normal healthy origin, by at least about 10%, about 20%, about 25%, about 30%, preferably at least about 40%, about 50%, more preferably at least about 60%, about 70%, or about 90%, about 100%, about 150%, or about 200%, or greater.

### Prognosis, Stage and Monitoring of Coronary Artery Disease

In one aspect, the present invention provides methods for determining onset, prognosis and stage of CAD based on examining the expression levels of a suitable, e.g., lipoprotein-modulating GALNT, e.g., mouse or human GALNT2, nucleic acid, polypeptide and/or the functional fragments thereof. As used herein, prognosis refers to the prediction of the probable course and outcome of a disease.

In general, the methods used for prognosis or stage of CAD involve comparison of the amount of a suitable GALNT in a sample of interest with that of a control sample to detect relative differences in the expression levels of the GALNT. The difference can be measured qualitatively and/or quantitatively. The control sample can be CAD-free or normal sample, or the sample known not to progress, or the sample known to progress.

Also as used herein, the CAD stage refers to the sequence of the events, in which the CAD develops and causes symptoms. In addition, staging is a process used to describe how advanced the CAD state is in a patient. Methods of the present invention are useful in assaying the staging of CAD. The staging can be accomplished by determining the expression levels of a suitable GALNT relative to a reference level. The reference level can be that from CAD-free, healthy samples, or CAD samples at different stages in disease development.

The present invention further provides methods of monitoring CAD progression or recurrence in a subject by measuring over time the expression levels of a suitable GALNT nucleic acid or polypeptide or the functional fragments thereof.

In one embodiment, the methods include a) determining at a first time point the expression level of a suitable GALNT nucleic acid molecule in the subject; b) determining at a subsequent time point the expression of the GALNT nucleic acid in the subject; and c) comparing the expression level at the first time point with that at the subsequent time point, wherein a significant change in the expression level is an indication of the onset, progression, or regression of the CAD.

In another embodiment, the methods include a) determining at a first time point the expression level of a GALNT polypeptide molecule in the subject; b) determining at a subsequent time point the expression of the GALNT polypeptide in the subject; and c) comparing the expression level at the first time point with that at the subsequent time point, wherein a significant change in the expression level is an indication of the onset, progression, or regression of the CAD.

Increased expression levels of the GALNT nucleic acid or polypeptide or the functional fragments thereof at the subsequent time point relative to the earlier time point indicate that the disease is progressing to a more severe stage. In contrast, reduced expression levels at the subsequent time point indicate that the disease is progressing to a less severe stage.

### Efficacy of Therapy and Therapeutic Compound

In another aspect, the present invention also provides methods that permit assessment and/or monitoring of a patient who will be likely to benefit from both traditional and non-traditional treatments and therapies for CADs. An advantage of the present invention is the ability to monitor, screen over time, those patients who can benefit from one, or several, of the available therapies over time to determine how the patient is faring from the treatment(s), whether a change, alteration, or cessation of treatment is warranted, or whether the patient's disease state or stage has progressed.

The identification of a correct patient for a particular therapy according to this invention can provide an increased efficacy of the treatment and can avoid subjecting the patient to unwanted and life-threatening side effects of the therapy. The ability to monitor a patient undergoing a course of therapy using the methods of the present invention can determine whether the patient is adequately responding to the therapy over time, to determine whether dosage or amount or mode of delivery should be altered or adjusted, and to ascertain whether the patient is improving during therapy, or is regressing or is entering a more severe or advanced stage of disease.

A method of monitoring according to this invention reflects the serial, or sequential, testing or analysis of a patient afflicted with a CAD or atherosclerotic condition by testing or analyzing the patient's body fluid sample over a period of time, such as during the course of treatment or therapy, or during the course of the patient's disease. For instance, in serial testing, the same patient provides a body fluid sample, e.g., serum or plasma, or has a sample taken for the purpose of observing, checking, or examining the expression levels of a suitable GALNT nucleic acid or polypeptide or the functional fragments thereof in the patient by measuring the GALNT levels during the course of treatment, and/or during the course of the disease, according to the methods of the invention.

Similarly, a patient can be screened over time to assess the GALNT levels in a biological sample for the purposes of determining the status of his or her disease and/or the efficacy, reaction, and response to the treatment or therapy that he or she is undergoing. It will be desirable that one or more biological samples are optimally taken from a patient prior to a course of treatment or therapy, or at the start of the treatment or therapy, to assist in the analysis and evaluation of patient progress and/or response at one or more later points in time during the period that the patient is receiving treatment and undergoing clinical and medical evaluation.

Levels can be monitored over a period of days, weeks, months, years, or various intervals thereof. The patient's body fluid sample, e.g., a serum or plasma sample, is collected at intervals, as determined by the practitioner, such as a physician or clinician, to determine the GALNT, plasma lipoproteins including HDL levels in the patient compared to the levels in normal individuals over the course of treatment or disease. For example, patient samples can be taken and monitored every month, every two months, or combinations of one, two, or three month intervals according to the invention. Quarterly, or more frequent monitoring of patient samples, is advisable. The levels found in the patient are compared with the levels in normal individuals, and with the patient's own levels obtained from prior testing periods, to determine treatment or disease progress or outcome.

A reduction in suitable GALNT levels over time, indicating an increase in plasma HDL-C, is indicative of treatment progress or efficacy, and/or disease improvement, remission, and the like.

### Kits

The present invention also provides for kits that contain the necessary reagents for detection of the expression levels (either nucleic acid or polypeptide level) of a suitable GALNT in a biological sample. Reagents can include specific probes/primers and antibodies as described supra. Kits can also contain a control/reference value or a set of control/reference values indicating normal and various clinical progression stages of disease. In a preferred embodiment, the control/reference value or a set of control/reference values are indicative of normal and various clinical progression stages of a CAD. Moreover, kits can contain a positive control, and/or a negative control for comparison with the test sample. The negative control can contain a sample that does not have a GALNT nucleic acid or polypeptide. The positive control can contain a sample that has various known levels of a suitable GALNT nucleic acid or polypeptide. Kits can also contain instructions for conducting the assays and for interpreting the results. For antibody-based kits, the kits can comprise, for example: (1) a first antibody (e.g., attached to a solid support) that binds to the GALNT polypeptide or functional fragments thereof; and, optionally, (2) a second, different antibody that binds to either the GALNT polypeptide, epitope thereof, or the first antibody and is conjugated to a detectable label. For oligonucleotide-based kits, the kits can comprise, for example: (1) an oligonucleotide, e.g., a detectably labeled oligonucleotide that hybridizes to a suitable GALNT nucleic acid sequence or (2) a pair of primers useful for amplifying the GALNT nucleic acid molecule. The kits can also comprise, e.g., a buffering agent, a preservative, or a protein stabilizing agent. The kits can further comprise components necessary for detecting the detectable label (e.g., an enzyme or a substrate). The kits can also contain a control sample or a series of control samples that can be assayed and compared to the test sample. Each component of the kits can be enclosed within an individual container and all of the various containers can be within a single package, along with instructions for interpreting the results of the assays performed using the kits.

Such kits can be used to determine whether a subject is suffering from or at an increased risk of developing a CAD or atherosclerotic condition. Furthermore, such kits can be used to determine the prognosis, stage, or monitoring the progression of a CAD or atherosclerotic condition. Furthermore, such kits can be used for drug screening or for selection of treatment for a CAD or atherosclerotic condition.

### Pharmaceutical Compositions

In another aspect, the present invention provides a composition, e.g., a pharmaceutical composition, containing one or a combination of a HDL modulating agent (e.g., monoclonal antibodies, or antigen-binding portion(s), antisense, siRNA, low molecular weight molecules), of the present invention, formulated together with a pharmaceutically acceptable carrier. Such compositions may include one or a combination of (e.g., two or more different) binding molecules. For example, a pharmaceutical composition of the invention can comprise a combination of antibodies or agents that bind to different epitopes on the target antigen or that have complementary activities.

Pharmaceutical compositions of the invention also can be administered in combination therapy, i.e., combined with other agents. For example, the combination therapy can include HDL modulating agent combined with at least one other cholesterol-reducing agent. Examples of therapeutic agents that can be used in combination therapy are described in greater detail below in the section on uses of the agents of the invention.

As used herein, “pharmaceutically acceptable carrier” includes any and all solvents, dispersion media, coatings, antibacterial and antifungal agents, isotonic and absorption delaying agents, and the like that are physiologically compatible. The carrier should be suitable for intravenous, intramuscular, subcutaneous, parenteral, spinal or epidermal administration (e.g., by injection or infusion). Depending on the route of administration, the active compound may be coated in a material to protect the compound from the action of acids and other natural conditions that may inactivate the compound.

The pharmaceutical compounds of the invention may include one or more pharmaceutically acceptable salts. A “pharmaceutically acceptable salt” refers to a salt that retains the desired biological activity of the parent compound and does not impart any undesired toxicological effects (see e.g., Berge, S. M., et al., 1977 J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and di-carboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A pharmaceutical composition of the invention also may include a pharmaceutically acceptable anti-oxidant. Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as, aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, one can include isotonic agents, for example, sugars, polyalcohols such as mannitol, sorbitol, or sodium chloride in the composition. Prolonged absorption of the injectable compositions can be brought about by including in the composition an agent that delays absorption for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the methods of preparation are vacuum drying and freeze-drying (lyophilization) that yield a powder of the active ingredient plus any additional desired ingredient from a previously sterile-filtered solution thereof.

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.01 percent to about ninety-nine percent of active ingredient, from about 0.1 percent to about 70 percent, or from about 1 percent to about 30 percent of active ingredient in combination with a pharmaceutically acceptable carrier.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of sensitivity in individuals.

For administration of the HDL modulating agent, e.g., an antibody, the dosage ranges from about 0.0001 to 100 mg/kg, and more usually 0.01 to 5 mg/kg, of the host body weight. For example dosages can be 0.3 mg/kg body weight, 1 mg/kg body weight, 3 mg/kg body weight, 5 mg/kg body weight or 10 mg/kg body weight or within the range of 1-10 mg/kg. An exemplary treatment regime entails administration once per week, once every two weeks, once every three weeks, once every four weeks, once a month, once every 3 months or once every three to 6 months. Dosage regimens for an antibody include 1 mg/kg body weight or 3 mg/kg body weight by intravenous administration, with the antibody being given using one of the following dosing schedules: every four weeks for six dosages, then every three months; every three weeks; 3 mg/kg body weight once followed by 1 mg/kg body weight every three weeks.

In some methods, two or more binding molecules (e.g., monoclonal antibodies) with different binding specificities are administered simultaneously, in which case the dosage of each antibody administered falls within the ranges indicated. The HDL modulating agent is usually administered on multiple occasions. Intervals between single dosages can be, for example, weekly, monthly, every three months or yearly. Intervals can also be irregular as indicated by measuring blood levels of HDL modulating agent (e.g., antibody) in the patient. In some methods, dosage is adjusted to achieve a plasma concentration of the antibody of about 1-1000 μg/ml and in some methods about 25-300 μg/ml.

Alternatively, HDL modulating agent can be administered as a sustained release formulation, in which case less frequent administration is required. Dosage and frequency vary depending on the half-life of the HDL modulating agent in the patient. For example, with antibodies, human antibodies show the longest half-life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated or until the patient shows partial or complete amelioration of symptoms of disease. Thereafter, the patient can be administered a prophylactic regime.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A “therapeutically effective dosage” of HDL modulating agent of the invention results in a decrease in severity of disease symptoms (e.g., a decrease in plasma cholesterol, or a decrease in a symptom of a cholesterol-related disorder), an increase in frequency and duration of disease symptom-free periods, or a prevention of impairment or disability due to the disease affliction.

A composition can be administered by one or more routes of administration using one or more of a variety of methods known in the art. As will be appreciated by the skilled artisan, the route and/or mode of administration will vary depending upon the desired results. Routes of administration for HDL modulating agent include, but are not limited to intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Alternatively, a HDL modulating agent can be administered by a nonparenteral route, such as a topical, epidermal or mucosal route of administration, for example, intranasally, orally, vaginally, rectally, sublingually or topically.

The active compounds can be prepared with carriers that will protect the compound against rapid release, such as a controlled release formulation, including implants, transdermal patches, and microencapsulated delivery systems. Biodegradable, bio compatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

Therapeutic compositions can be administered with medical devices known in the art. For example, in one embodiment, a therapeutic composition of the invention can be administered with a needleless hypodermic injection device, such as the devices shown in U.S. Pat. No. 5,399,163; 5,383,851; 5,312,335; 5,064,413; 4,941,880; 4,790,824 or 4,596,556. Examples of well known implants and modules useful in the present invention include: U.S. Pat. No. 4,487,603, which shows an implantable micro-infusion pump for dispensing medication at a controlled rate; U.S. Pat. No. 4,486,194, which shows a therapeutic device for administering medicants through the skin; U.S. Pat. No. 4,447,233, which shows a medication infusion pump for delivering medication at a precise infusion rate; U.S. Pat. No. 4,447,224, which shows a variable flow implantable infusion apparatus for continuous drug delivery; U.S. Pat. No. 4,439,196, which shows an osmotic drug delivery system having multi-chamber compartments; and U.S. Pat. No. 4,475,196, which shows an osmotic drug delivery system. These patents are incorporated herein by reference. Many other such implants, delivery systems, and modules are known to those skilled in the art.

In certain embodiments, the HDL modulating agent of the invention can be formulated to ensure proper distribution in vivo. For example, the blood-brain barrier (BBB) excludes many highly hydrophilic compounds. To ensure that the therapeutic compounds of the invention cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., V. V. Ranade, 1989 J. Cline Pharmacol. 29:685). Exemplary targeting moieties include folate or biotin (see, e.g., U.S. Pat. No. 5,416,016 to Low et al.); mannosides (Umezawa et al., 1988 Biochem. Biophys. Res. Commun. 153:1038); antibodies (P. G. Bloeman et al., 1995 FEBS Lett. 357:140; M. Owais et al., 1995 Antimicrob. Agents Chemother. 39:180); surfactant protein A receptor (Briscoe et al., 1995 Am. J. Physiol. 1233:134); p 120 (Schreier et al., 1994 J. Biol. Chem. 269:9090); see also K. Keinanen; M. L. Laukkanen, 1994 FEBS Lett. 346:123; J. J. Killion; I. J. Fidler, 1994 Immunomethods 4:273

### EXAMPLES

There is strong evidence that raising the plasma levels of functional HDL decreases the risk of coronary artery disease (Gotto, A. et al., J. Am. Coll. Cardiol., 43:717-724, 2004). The role of human GALNT2 in regulating plasma HDL-C levels, which was previously unknown, is disclosed herein. Using a combination of mouse genetics, human genetics, mouse-comparative genomics, and bioinformatics methods, GLANT2 was found as a gene regulating plasma HDL-C levels. The role of this GALNT in modulating plasma lipoprotein and HDL-C indicates its usefulness in screening for agents that inhibit these GALNTs, making them useful in treating CADs or atherosclerotic conditions, as well as their usefulness as, for example, biological markers for CADs, atherosclerotic conditions, or a susceptibility to CADs or atherosclerotic conditions.

Quantitative trait locus (QTL) analysis is a means for finding novel genes that regulate complex traits (Abiola O et al., 2003, Nature Rev Genet 4:911-916). QTL analysis is particularly important for biomedical research because QTLs detected in mouse models of disease often predict the location of human disease QTLs, suggesting that genetic analysis using mouse models can potentially identify genes that are important for human disease (Abiola O et al., 2003, Nature Rev Genet. 4:911-916; Rollins J et al., 2006, Trends Cardiovasc Med 16:220-234; Chen et al., 2007, Cell Metab 6: 164-179).

A QTL for plasma HDL-C levels was found on mouse distal chromosome (Chr) 8 from crosses B6xCAST and B6xCASA (Rollins J et al., 2006, Trends Cardiovasc Med 16:220-234). We found from our mouse genome-wide association studies that polymorphisms of Galnt2 were associated with HDL-C levels in mouse populations (P=3.5×10⁻⁵), according to the method described in Pletcher et al., PLoS Biol. 2:e393 (2004).

Haplotype analysis also showed that Galnt2 is a candidate gene based on the above two crosses (Table 1). SNP positions within the Galnt2 gene on chromosome 8 are highlighted in Table 1, where “bp” is basepair. (C57BL/6J) B6 and CAST have different haplotypes and SNP data within Galnt2 for CASA is not available. However, according to the mouse Phenome Database, CAST and CASA have the same haplotypes in regions surrounding this gene. Based on this evidence and the relatedness of CAST and CASA (both strains were inbred from the same group of wild-trapped Castaneus at the Jackson Laboratory), it can be assumed that CASA and B6 haplotypes are different within the Galnt2 gene. Accordingly, the allelic variations in Galnt2 and the association with altered HDL levels suggest a role for this gene in HDL metabolism. As such, expression of this gene is useful as a biomarker for CAD caused by lowered HDL levels.

TABLE 1
Haplotype comparison among different mouse strains

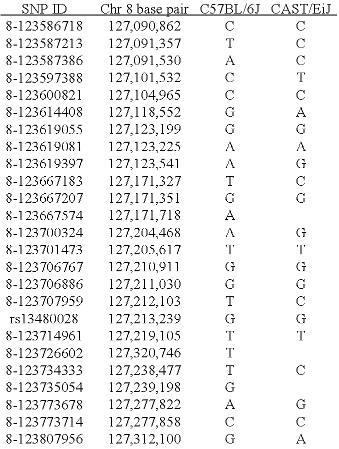

B6 and CAST mice were fed a chow or a high fat diet. Mice, at 10-11 weeks of age, were fasted 4 hours before blood samples were taken. Plasma was placed into a fresh tube and frozen at −20° until analyzed. Plasma samples were thawed and analyzed within a week of being collected. Plasma lipoprotein concentrations from each blood sample were measured directly, using an enzymatic reagent kit (no. 650207, Beckman Coulter) according to manufacturer's recommendations on the Synchron CX Delta System (Beckman Coulter).

The expression of GALNT2 was investigated in two strains of mice having different HDL levels. Fold changes and q-values for GALNT2 between B6 and CAST are shown in the Table 2. The fold changes indicated are in B6 compared to CAST. The data show that GALNT2 is down-regulated in B6, the high allele strain. Thus, GALNT2 expression correlates with HDL, showing a Pearson coefficient of −0.3801 (p=0.019). This correlation validates the negative fold change seen in B6 compared to CAST. Based on this relationship, inhibiting GALNT2 is predicted to raise HDL. Thus, methods comprising inhibition of GALNT2 can be used to treat CAD or atherosclerotic disease.

TABLE 2
Galnt2 gene expression change in liver (B6 vs. CAST)

| Sex | Diet | Fold change | q-value |
|---|---|---|---|
| Female | Chow | −1.8 | 0.00004 |
| Female | High fat | −1.2 | 0.06738 |
| Male | Chow | −1.4 | 0.00152 |
| Male | High fat | −1.5 | 0.00348 |

The role of GALNT2 in HDL modulation in humans is further evidenced by the Novartis-Broad collaboration study which showed that polymorphisms of four SNPs (rs2144300, rs 17315646, rs4846914, and rs10127775) in GALNT2 are associated with plasma HDL levels in humans (P=1.4×10⁻⁴ to 3.4×10⁻⁴).

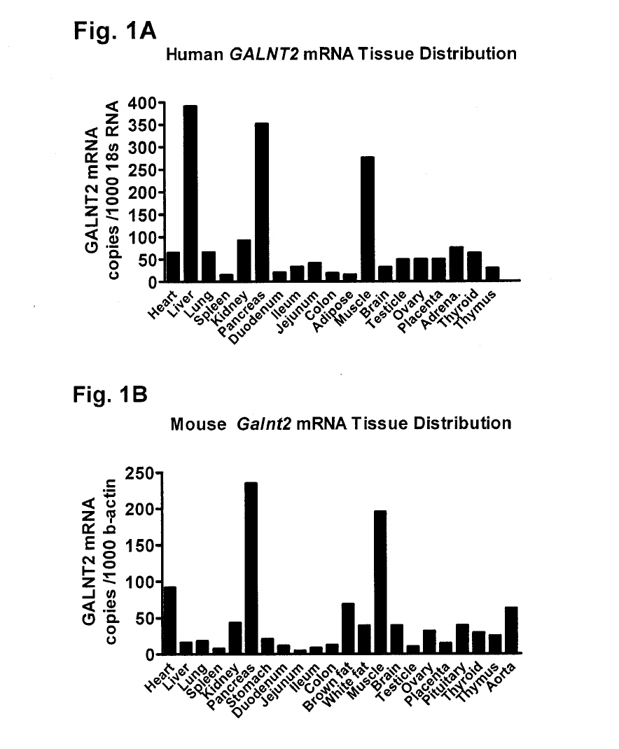

The gene expression of GALNT2 was examined in both mouse and human tissues using real-time quantitative PCR. As shown in FIG. 1, GALNT2 is highly expressed in the liver, pancreas, and muscle in the human (FIG. 1A), and in the pancreas and muscle in the mouse (FIG. 1B). Expression levels were normalized relative to β-actin mRNA in mouse tissues and to 18s RNA in human tissues.

1. Use of an isolated antibody or functional fragment thereof, comprising an antigen-binding region that is specific for an epitope of a polypeptide encoded by a GALNT gene, wherein the antibody or functional fragment binds to a surface receptor on a cell, and prevents or ameliorates development of a HDL-associated disease.

2. The use according to claim 1, wherein the GALNT gene is a GALNT2 gene.

3. A method for treating a HDL-associated disease comprising administering to a subject an effective amount of the antibody or functional fragment thereof, according to claim 1.

4. A pharmaceutical composition comprising an antibody or functional fragment according to claim 1 and a pharmaceutically acceptable carrier or excipient therefore.

5. A method for treating a HDL-associated disease comprising administering to a subject in need thereof an effective amount of the pharmaceutical composition according to claim 4.

6. Use of an isolated antibody or functional fragment thereof for the preparation of a medicament for the treatment of a HDL-associated disease, wherein the antibody or functional fragment comprising an antigen-binding region that is specific for an epitope of a polypeptide encoded by a GALNT gene.

7. The use of claim 6, wherein the GALNT gene is a GALNT2 gene.

8. A transgenic animal carrying a gene encoding an antibody or functional fragment thereof according to claim 1.

9. A method for treating a coronary artery disease or atherosclerotic condition comprising inhibiting the expression and/or activity of a GALNT.

10. The method of claim 9, wherein the GALNT is human GALNT2.

11. The method of claim 9, wherein the step of inhibiting the expression and/or activity of a carboxylesterase further comprises inhibiting the activity using an isolated antibody or functional fragment thereof comprising an antigen-binding region that is specific for an epitope of a polypeptide encoded by a carboxylesterase gene.

12. The method of claim 12, wherein the isolated antibody or functional fragment thereof comprising an antigen-binding region binds to a surface receptor on a cell and prevents or ameliorates the development of a HDL-associated disease.

13. A method for detecting a coronary artery disease or susceptibility to a coronary artery disease comprising detecting the alleles of the human GALNT2 gene that is indicative of a coronary artery disease or atherosclerotic condition.

14. A method for determining the efficacy of treating a coronary artery disease or atherosclerotic condition comprising the treatment of a coronary artery disease or an atherosclerotic condition, and comparing the level of the GALNT with a reference such that the efficacy of treating the coronary artery disease or atherosclerotic condition is determined.

15. The method of claim 14, wherein the GALNT is human GALNT2.

16. A method of identifying an agent useful for treating a coronary artery disease or an atherosclerotic condition, wherein inhibition of the GALNT induces increased plasma HDL-C levels, comprising contacting a biological sample with a candidate agent and determining the level of HDL-C in the sample before and after contact with the candidate agent, wherein an increase in HDL-C is indicative of an agent that is useful for treating a coronary artery disease or an atherosclerotic condition.

17. A method for identifying an agent useful for treating a coronary artery disease or atherosclerotic condition comprising contacting a GALNT with a candidate agent in the presence of a known GALNT substrate, wherein a decrease in the GALNT activity of the GALNT identifies the candidate agent as an agent useful for treating a coronary artery disease or an atherosclerotic condition.

18. The method of claim 17, wherein the GALNT is mouse or human GALNT2.

19. The method of claim 17, wherein the contacting step is performed in a cultured cell.

20. The method of claim 17, wherein the contacting step is performed in vivo.

21. The method of claim 17, wherein the GALNT is endogenous or exogenous.

22. A method for modulating a HDL-associated disease comprising administering a HDL modulating agent that elevates HDL-C levels in a subject.

23. The method of claim 22, wherein the HDL-associated disease is selected from the group consisting of atherosclerosis, lipid disorders, Alzheimer's disease, excessive oxidative stress, endothelial dysfunction, obesity, chronic renal disease, type II diabetes and insulin resistance.

24. The method of claim 23, wherein the lipid disorder is selected from the group consisting of: elevated cholesterol, dyslipidemic syndrome, elevated triglycerides, dyslipidemia, dyslipoproteinemia, hyperlipidemia, familial hypercholesterolemia, and familial hypertriglyceridemia.

25. The method of claim 22, wherein the agent inhibits the activity or decreases the expression of human GALNT2.

26. The method of claim 25, wherein the HDL modulating agent is selected from the group consisting of a low molecular weight molecule, an antisense oligonucleotide, siRNA, shRNA and an antibody.

27. The method of claim 22, wherein the HDL-associated disease is any disease in which the HDL-C levels in the subject is below the accepted normal HDL-C level.

28. The method of claim 22, wherein the HDL-associated disease is any disease in which the HDL-C levels in the subject is below the normal HDL-C level of the related population.

29. The method of claim 22, wherein the HDL modulating agent is administered with a pharmaceutically acceptable carrier.
